(19) United States
(12) Patent Application Publication
(10) Pub. No.: US 2009/0170177 A1
(43) Pub. Date: Jul. 2, 2009

(54) STEM CELL TRANSFECTION METHOD

(75) Inventors: Mei-Ling Ho, Kaohsiung City (TW); Gwo-Jaw Wang, Kaohsiung City (TW); Je-Ken Chang, Kaohsiung City (TW); Yan-Hsiung Wang, Kaohsiung City (TW)

Correspondence Address: THOMAS, KAYDEN, HORSTEMEYER & RISLEY, LLP, 600 GALLERIA PARKWAY, S.E., STE 1500, ATLANTA, GA, 30339-5994, US

(73) Assignee: KAOHSIUNG MEDICAL UNIVERSITY, Kaohsiung City (TW)

(21) Appl. No.: 12/106,436

(22) Filed: Apr. 21, 2008

(30) Foreign Application Priority Data
Dec. 31, 2007 (TW) 96151439

(51) Int. Cl. C12N 13/00

(52) U.S. Cl. 435/173.6

(57) ABSTRACT

Stem cell transfection method. The stem cell infection method of the invention comprises providing a stem cell; positioning the stem cell at a buffer, wherein the buffer contains a foreign material; electroporating the stem cell in the buffer; and culturing the stem cell.

### BACKGROUND OF THE INVENTION

1. Field of the Invention

The present invention relates to a cell transfection, and in particular relates to a stem cell electroporation transfection.

2. Description of the Related Art

The characteristics of stem cells are that they are clonogenic, self-renewing and can give rise to specialized cell types. There are two kinds of stem cells: embryonic stem cells and adult stem cells, which are defined according the isolated tissue source. Embryonic stem cells are pluripotent and can become all cell types of the body. Generally, adult stem cells are limited to the number and the type of differentiated cells types that they can become. For cell-based tissue regeneration, a potential advantage of using stem cells from an adult is that the patient's own cells can be expanded in culture and then reintroduced into the patient so that the cells would not be rejected by the immune system. Mesenchymal stem cells are isolated from mesodermal organs, such as bone marrow, umbilical cord blood, and fat tissue. They have the ability to differentiate into mesodermal cell lineages under appropriate culturing conditions, such as muscle, bone, cartilage, and fat. Therefore, mesenchymal stem cells are suitable cell sources for tissue regeneration and gene therapy.

Adult mesenchymal stem cells', due to its biology and ability, can be used for gene therapy providing a great potential for tissue regeneration. Recently, stem cells have been successfully transduced with therapeutic genes via viral vehicles. However, the risk of inducing toxicity, immune and inflammatory responses increased by the virus (viral vehicles). In addition, viral-based methods are difficult to set up due to its time-consuming nature and requirement for specific safety conditions, especially with human cells. Non-viral delivery methods include native DNA, liposome, cation polymer, and electroporation. However, gene deliveries using non-viral methods are less efficient than viral mediated DNA delivery. Typically, transfection efficiency is limited to 20%-25%. Still, non-viral methods have several advantages, including cheaper manufacture costs, none or weak immunogenic response during repeat administration

Thus, to overcome the disadvantage of the viral-based transfection method, a highly efficient and safe non-viral transfection method is needed.

### BRIEF SUMMARY OF INVENTION

The invention provides a stem cell transfection method. The stem cell infection method of the invention comprises: providing a stem cell; positioning the stem cell at a buffer, wherein buffer contains a foreign material; electroporating the stem cell in the buffer to transfect the foreign to the stem cell; and culturing the stem cell. The stem cell transfection method of the invention has high transfect efficiency, and does not suppress differentiation and proliferation of the stem cell.

A detailed description is given in the following embodiments with reference to the accompanying drawings.

### BRIEF DESCRIPTION OF DRAWINGS

The present invention can be more fully understood by reading the subsequent detailed description and examples with references made to the accompanying drawings, wherein:

FIG. 1 shows the transfection efficiency of the electroporation programs in Example 1;

FIGS. 2a-2b show increase in the number of the stem cells at sub-G1 phase after electroporation;

FIG. 3 shows release of lactate dehydrogenase from the stem cell to the outside after electroporation;

FIG. 4 shows that EGFP expression is increased dependent upon increase of plasmid concentration;

FIGS. 5a-5b show that the electroporated hADSCs differentiates into the mineralized nodule and oil body;

FIG. 5c shows detection of EGFP expression by fluorescence microscopy in the osteoblastic cell after osteoinduction; and

FIG. 6 shows that the electroporated stem cell proliferates and differentiates in animals.

### DETAILED DESCRIPTION OF INVENTION

The following description is of the best-contemplated mode of carrying out the invention. This description is made for the purpose of illustrating the general principles of the invention and should not be taken in a limiting sense. The scope of the invention is best determined by reference to the appended claims.

A stem cell transfection method is provided. The stem cell transfection method of the invention comprises: providing a stem cell; positioning the stem cell at a buffer, wherein the buffer contains a foreign material; electroporating the stem cell in the buffer; and culturing the stem cell.

The term “stem cell” is used herein to refer to a mammalian cell that has the ability both to self-renew, and to generate differentiated progeny (see Morrison et al. (1997) Cell 88:287-298). Generally, stem cells also have one or more of the following properties: an ability to undergo asynchronous, or symmetric replication, where the two daughter cells after division can have different phenotypes; extensive self-renewal capacity; capacity for existence in a mitotically quiescent form; and clonal regeneration of all of the tissue, for example the ability of hematopoietic stem cells to reconstitute all hematopoietic lineages. The stem cell of the invention includes, but are not limited to, a blood stem cell, an adipose stem cell, a bone marrow mesenchymal stem cell, a mesenchymal stem cell, a neural stem cell, a skin stem cell, an embryonic stem cell, an endothelial stem cell, a hepatic stem cell, a pancreatic stem cell, an intestinal epithelium stem cell, or a germ stem cell.

Firstly, a stem cell is washed with a phosphate buffer saline, suspended in a suspension buffer, and then one or more foreign material(s) is added. The suspension buffer can be a common electroporation buffer.

The term “foreign material” of the invention can be any materials excluding the cell itself. For example, foreign DNA, RNA, gene, plasmid, vector, or peptide.

In one embodiment, to obtain a stem cell producing insulin, a plasmid containing insulin gene can be sued as the foreign material and transfected into the stem cell.

Next, an electroporation process is performed to transfect the foreign material to the stem cell. After the electroporation process, the transfected stem cell is cultured at a suitable environment.

As used herein, the term “electroporation” means the temporary creation of holes or aqueous pores in the surface of a cell membrane by an applied electrical potential and through which therapeutic agents may pass into the cell. Electroporation is now widely used in biology, particularly for transfection studies, where plasmids, DNA fragments and other genetic material are introduced into living cells. During electroporation pulsing, molecules which are not normally membrane permeant are able to pass from the extracellular environment into the cells during the period of induced reversible membrane permeabilization. The permeabilized state is caused by the generation of an electrical field in the cell suspension or tissue of sufficient field strength to perturb the cell surface membrane's proteolipid structure. This perturbation is believed to be due to both a constituent charge separation and the effect of viscoelastic compression forces within the membrane and it's sub-adjacent cytoskeletal structures. The result is localized membrane thinning. At a critical external field strength, pores or small domains of increased permeability are formed in the membrane proteolipid bi-layer.

The electroporation program of the invention can be 900-1800 voltage, 20 ms, and 1-2 pulse, preferably, 1500 voltage, 20 ms, and 1 pulse.

The transfection efficiency of the invention exceeds 30%, preferably, about 60-80%, and the transfection efficiency can be increased dependant upon increasing the concentration of the foreign material. Additionally, the stem cell transfection method of the invention does not suppress or affect the differentiation of the stem cell. Specifically, the stem cell still maintains the differentiation ability after electroporation.

### EXAMPLE

### Example 1

### Transfection of the Stem Cell

Subconfluent hADSCs (human adipose tissue-derived mesenchymal stem cells) and hBMSCs (human bone marrow mesenchymal stem Cells) were harvested and washed with phosphate-buffered saline (PBS), resuspended in Resuspension buffer R at a density about 1×107 cells/ml, and then incubated with pCMV-EGFP-N1 plasmids containing EGFP gene. Then, electroporation was performed with MP-100 (Digital Bio Technology Co., Ltd. Korea) at room temperature by different programs. Program 1 (P1): 900 voltage, 20 ms, one pulse. Program 2 (P2): 900 voltage, 20 ms, two pulses. Program 3 (P3): 1500 voltage, 20 ms, one pulse. Program 4 (P4): 1500 voltage, 20 ms, two pulse. Program 5 (P5): 1800 voltage, 20 ms, one pulse. After electroporation, stem cells were distributed into 35-nun cell culture dishes and placed at 37° C. in a 5% CO₂ humidified atmosphere. The stem cells were harvested for 24 hours and washed twice with PBS.

### Example 2

### Analysis of the Transfection Efficiency

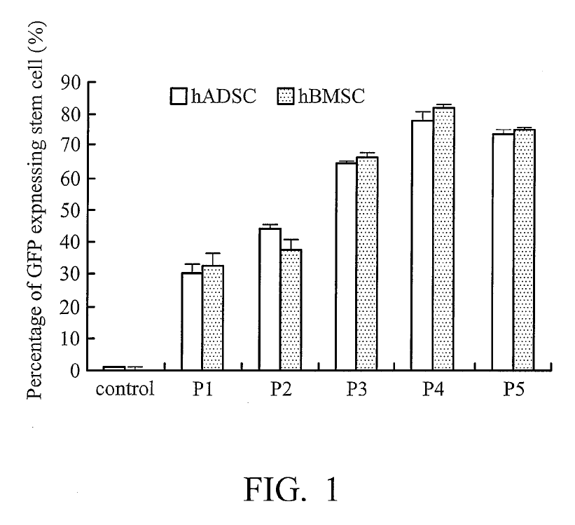

After the electroporation of Example 1, EGFP positive cells (EGFP+ cells) were analyzed by flow cytomatric analysis. For the control group, electroporation was not performed, and the stem cells were only cultured at K-NAC medium (Invitrogen) in a 5% CO₂ humidified atmosphere. Referring to FIG. 1, the EGFP+ cells represented more than 30% of transfected hADSCs or hBMSCs with all programs. In hADSCs, the EGFP+ cells ratio of P1, P2, P3, P4 and P5 program was 30.5%±2.7%, 44.4%±1.2%, 64.8%±0.8%, 77.8%±2.9%, and 73.9%±1.6%, respectively. In hBMSCs, the EGFP+ cells ratio of P1, P2, P3, P4 and P5 program was 32.7%±3.8%, 39.8%±2.8%, 67.1%±0.9%, 82.5%±0.6%, and 74.9%±0.9%. FIG. 1 indicated that the transfection efficiency of all electroporation programs in Example 1 exceeded 30%.

### Example 3

### Effect of the Electroporation on Stem Cells

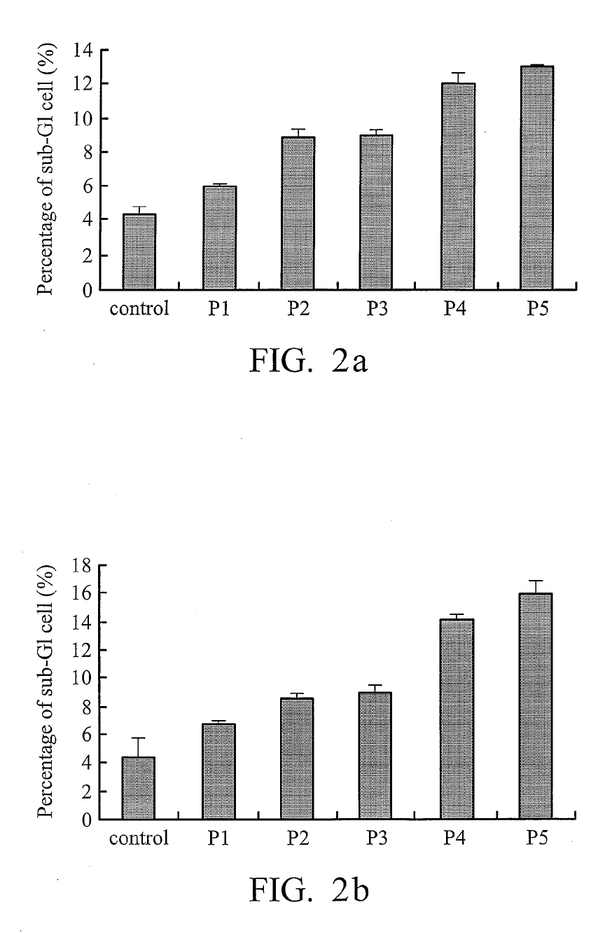

The same procedure carried out in Example 1 was repeated. hADSCs and hBMSCs were microporated at P1-P5 programs. After 24 hours, hADSCs and hBMSCs were fixed and stained with propidium iodide, respectively, and then analyzed by FACS analysis (Fluorescence-Activated Cell Sorter). The experiment was repeated three times independently, and the results were expressed as mean±S.D. Referring to FIG. 2a, After electroporation, the percentage of hADSCs at sub-G1 phase was increased. Referring to FIG. 2b, similarly, the percentage of hBMSCs at sub-G1 phase was increased.

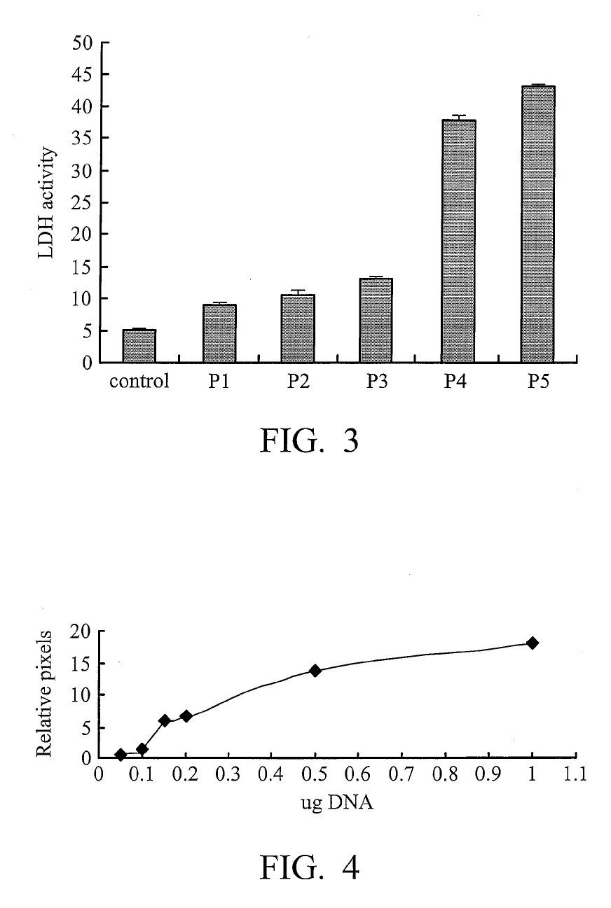

Additionally, the lactate dehydrogenase (LDH) activity was detected to infer the effect of the electroporation on hADSCs. Suitable processes for detecting LDH activity include, for example, those illustrated in references such Chang J K et al. Toxicology. 2006; 228:111-23. The experiment was repeated three times independently, and the results were expressed as mean±S.D. Referring to FIG. 3, after electroporation, the cell membrane of hADSCs was lightly damaged, so that LDH was released from the hADSCs through the damaged cell membrane. Compared with the control group, the LDH leakage of electroporated hADSCs was elevated by 8.9%±0.6% (P1), 10.6%±0.6% (P2), 12.9%±0.4% (P3), 37.8%±0.9% (P4) and 43.1%±0.5% (P5), respectively. The data indicates that the electroporation induces cytotoxic effects on hADSCs and the cytotoxic effects were increased based on voltage and pulse number. P3 program was the excellent transfection condition for hADSCs because of the higher transfection rate and lower cytotoxic effect.

### Example 4

### Effect of Plasmid Concentration on Transfection Efficiency

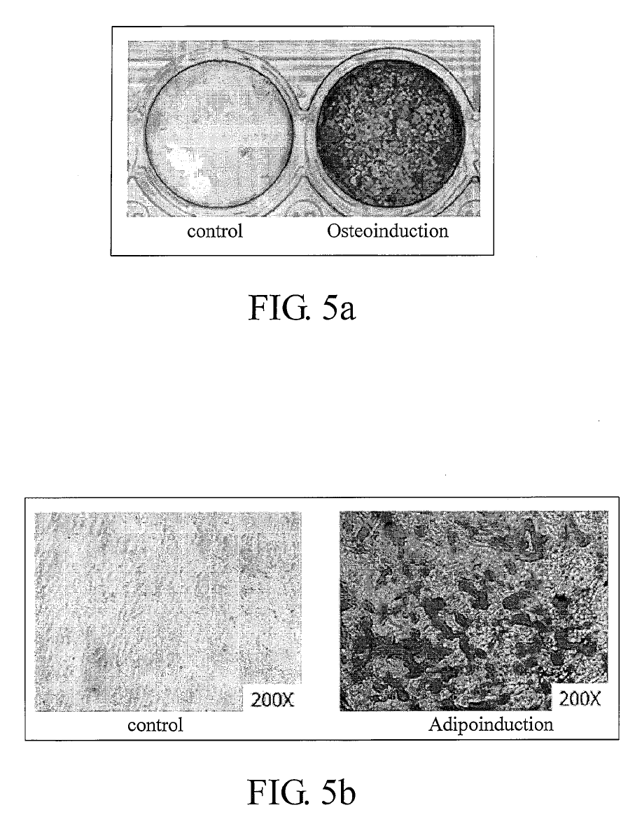

The same procedure carried out in Example 1 was repeated. hADSCs were transfected without or with different dosage (0.05, 0.1, 0.15, 0.2, 0.5 or 1 ug) pCMV-EGFP-N1 using P3 program, and then the electroporated hADSCs were cultured at 37° C. in a 5% CO₂ humidified atmosphere for 48 hours. The expression level of EGFP was determined by Western blot with anti-GFP antibody. The graph showed densitometry analysis of EGFP expression that was normalized against β-actin. Referring to FIG. 4, the EGFP expression was increased dependent upon increasing pCMV-EGFP-N1 concentration. For correlation, intensities of signals were compared with intensities of β-actin signals, and 0.15 μg of pCMV-EGFP-N1 was the most effective dose for EGFP expression.

### Example 5

### Effect of the Electroporation on Stem Cell Differentiation

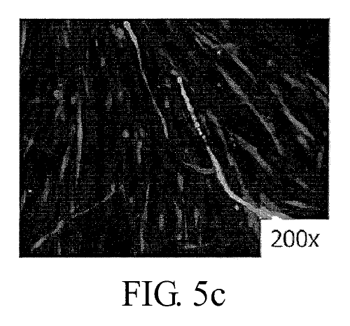

The same procedure carried out in Example 1 was repeated. hADSCs were transfected with pCMV-EGFP-N1. After 24 hours, hADSCs were cultured in a control medium or osteogenic medium for 14 days respectively, and then fixed and stained with propidium iodide. On the other hand, hADSCs were cultured in a control medium or adipogenic medium for 12 days, and then fixed and stained with oil red. The control medium was K-NAC medium with 5% FBS in 5% CO₂. Referring to FIG. 5a, the electroporated hADSCs were differentiated to form mineralized nodule in the osteogenic medium. Referring to FIG. 5b, the electroporated hADSCs were differentiated to form oil body in the adipogenic medium. Referring to FIG. 5c, the EGFP expression was detected by fluorescence microscopy in the osteoblastic cell after osteoinduction. FIGS. 5a-5c indicates that the stem cell transfection method of the invention does not affect the differentiation of the stem cell.

### Example 6

### Animal Experiment

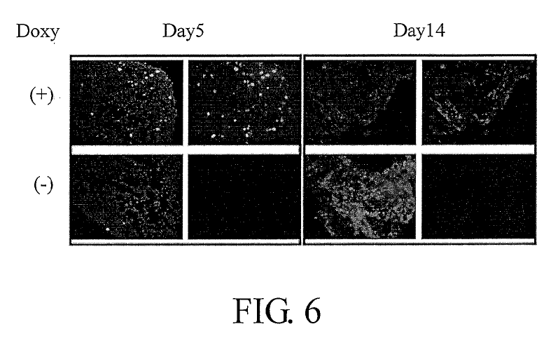

The same procedure carried out in Example 1 was repeated. hADSCs were co-transfected with pBI-EGFP and pTet-ON plasmid. After 24 hours, stem cells were trypsinized and then washed with PBS. After centrifugation, 10⁵ hADSCs were resuspended in cold serum free DMEM and mixed with an equal volume of cold Matrigel (10 μg/ml). A total volume of about 0.3 ml was subcutaneously injected into both back flanks of nude mice. Each mouse was implanted at six locations and divided into two experimental groups: Group 1 (control group) never received doxycycline (Doxy) with the drinking water, and Group 2 received Doxy with the drinking water (200 μg/ml). The drinking water contained 2.5% sucrose and water bottles wrapped with aluminum foil. The bottles of water were changed every 3 days. Mice were sacrificed at day 5 or day 14 after the injection. The Matrigel plugs were embedded in OCT compound, and quick-frozen in liquid nitrogen. The frozen materials were cut into 6- to 7-μm thick sections using a cryostat (Leica CM1900, Wetzlar, Germany). The sections were rehydrated in cold PBS. Nuclei were counterstained with DAPI (2 ng/ml) for 5 minuets and mounted. Images were acquired by microscope. The method of cell transplantation was illustrated in references such Glondu M et al. Oncogene. 2001; 20: 6920-6929. Referring to FIG. 6, the green fluorescence was detected in Group 2 which meant that the electroporated stem cell maintained the proliferation and differentiation in animal.

While the invention has been described by way of example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A stem cell transfection method, comprising
providing a stem cell;
positioning the stem cell at a buffer, wherein the buffer contains a foreign material;
electroporating the stem cell in the buffer to transfect the foreign to the stem cell, and
culturing the stem cell.

2. The stem cell transfection method as claimed in claim 1, wherein the program of the electroporating is 900-1800 voltage, and 20 ms.

3. The stem cell transfection method as claimed in claim 1, wherein the program of the electroporating is 1500 voltage.

4. The stem cell transfection method as claimed in claim 1, wherein the program of the electroporating is 1500 voltage, 20 ms, and 1 pulse.

5. The stem cell transfection method as claimed in claim 1, wherein the transfection efficiency of the stem cell transfection method exceeds 30%.

6. The stem cell transfection method as claimed in claim 1, wherein the transfection efficiency of the stem cell transfection method is about 60-80%.

7. The stem cell transfection method as claimed in claim 1, wherein the stem cell transfection method does not affect the differentiation of the stem cell.

8. The stem cell transfection method as claimed in claim 1, wherein the stem cell comprises a blood stem cell, an adipose stem cell, a bone marrow mesenchymal stem cell, a mesenchymal stem cell, a neural stem cell, a skin stem cell, an embryonic stem cell, an endothelial stem cell, a hepatic stem cell, a pancreatic stem cell, an intestinal epithelium stem cell, or a germ stem cell.

9. The stem cell transfection method as claimed in claim 1, wherein the stem cell is an adipose stem cell or bone marrow mesenchymal stem cell.

10. The stem cell transfection method as claimed in claim 1, wherein the foreign material comprises DNA, RNA, plasmid, vector, or peptide.
